SEQUENCE LISTING (2 sequences)

SEQ ID NO: 1 - protein, 447 aa, Artificial Sequence
  Heavy chain amino acid sequence of Pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 218 aa, Artificial Sequence
  Light chain amino acid sequence of Pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0226154 A1
(43) Pub. Date: Jul. 20, 2023

(54) COMPOSITIONS AND METHODS FOR TREATING CANCER

(71) Applicant: Trizell Ltd., West Drayton (GB)

(72) Inventors: Richard Philipson, Chinnor (GB); Seppo Ylä-Herttuala, Kuopio (FI)

(21) Appl. No.: 17/915,788

(22) PCT Filed: Mar. 30, 2021
(86) PCT No.: PCT/IB2021/000206

§ 371 Date: Sep. 29, 2022

Related U.S. Application Data
(60) Provisional application No. 63/002,168, filed on Mar. 30, 2020.

(51) Int. Cl. A61K 38/21; A61P 35/00

(52) U.S. Cl. CPC A61K 38/212; A61P 35/00; C12N 2710/10343

(57) ABSTRACT

The present disclosure includes compositions and methods for treating cancer, such as bladder cancer.

### CROSS REFERENCE TO RELATED APPLICATIONS

This application claims the benefit of U.S. Provisional Pat. Application No. 63/002,168 filed Mar. 30, 2020, the entire contents of which are hereby incorporated by reference.

### BACKGROUND

Bacillus Calmette-Guerin (BCG) is currently used to treat patients with high-grade non-muscle invasive bladder cancer (NMIBC). Most patients that receive BCG experience tumor recurrence over time, and many patients can develop high-grade NMIBC that is BCG-unresponsive. Radical cystectomy is another currently available treatment for high-grade NMIBC; however, the procedure is associated with high perioperative morbidity, and many patients are unwilling or unable to undergo the procedure. Various agents have been evaluated as salvage intravesical therapies after treatment with BCG; however, none to date have provided robust and durable responses in patients.

Thus, there is an unmet need for effective and safe treatments for patients with bladder cancers, particularly high-grade NMIBC.

### SUMMARY

The present disclosure provides, inter alia, compositions and methods for treating cancerous disorders (e.g., bladder cancer, e.g., high-grade non-muscle-invasive bladder cancer (NMIBC)). Disclosed herein, at least in part, are compositions (e.g., pharmaceutical compositions) comprising: (i) a recombinant adenoviral vector (e.g., a non-replicating recombinant adenoviral vector) encoding interferon α-2b, and (ii) [N-(3-cholamidopropyl)-N-(3-lactobionamidopropyl)]-cholamide (SYN3). The compositions (e.g., pharmaceutical compositions) disclosed herein can be used alone or in combination with other therapeutic agents, procedures, or modalities to treat or prevent cancerous disorders (e.g., bladder cancer, e.g., high-grade NMIBC).

In one aspect, the disclosure features a method of treating a high-grade non-muscle-invasive bladder cancer (NMIBC) in a subject, comprising administering a composition to the subject intravesically every three months for at least one year, wherein the composition comprises: (i) a non-replicating recombinant adenoviral vector (e.g., a type 5 non-replicating recombinant adenoviral vector) encoding interferon α-2b (IFN α-2b), and (ii) [N-(3-cholamidopropyl)-N-(3-lactobionamidopropyl)]-cholamide (SYN3), thereby treating NMIBC in the subject.

In some embodiments, the method further comprises performing an assay on the subject or on a sample from the subject, e.g., at about one year or longer after administration of the composition. In some embodiments, the assay indicates efficacy of the composition.

In one aspect, the disclosure features a method of evaluating or monitoring efficacy of a composition in a subject having or diagnosed with high-grade non-muscle-invasive bladder cancer (NMIBC), comprising performing an assay on the subject or on a sample from the subject, wherein the composition comprises: (i) a non-replicating recombinant adenoviral vector encoding interferon α-2b (e.g., a type 5 non-replicating recombinant adenoviral vector), and (ii) [N-(3-cholamidopropyl)-N-(3-lactobionamidopropyl)]-cholamide (SYN3), and wherein the subject has received intravesical administration of the composition every three months for at least one year.

In some embodiments, the method further comprises administering the composition to the subject. In some embodiments, the method further comprises acquiring the sample from the subject. In some embodiments, the sample comprises a blood sample (e.g., a plasma or serum sample), saliva sample, tissue sample, or urine sample. In some embodiments, the assay comprises performing cytology on the sample and/or cystoscopy on the subject. In some embodiments, cytology and/or cystoscopy are not performed.

In some embodiments, the method further comprises administering to the subject at least one additional therapeutic agent. In some embodiments, the at least one additional therapeutic agent comprises a checkpoint inhibitor. In some embodiments, the checkpoint inhibitor targets a checkpoint molecule chosen from PD-1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (e.g., TNFRSF14 or CD270), KIR, A2aR, MHC class I, MHC class II, GAL9, adenosine, or TGF (e.g., TGF beta). In some embodiments, the checkpoint inhibitor comprises or is an anti-PD-1 antibody or a fragment thereof, e.g., Pembrolizumab. In some embodiments, Pembrolizumab is administered intravenously about every three weeks or longer than every three weeks (e.g., every four weeks, every five weeks, every six weeks, or more).

In some embodiments, the subject has a complete response (CR), a partial response, or a non-response to treatment with the composition. In some embodiments, the subject does not exhibit high-grade recurrence after receiving treatment with the composition, e.g., for at least one year after administration of the composition. In some embodiments, the subject has an Eastern Cooperative Oncology Group status of 2 or less than 2. In some embodiments, the patient has high-risk NMIBC. In some embodiments, the patient has not been responsive to at least one prior treatment comprising Bacillus Calmette-Guerin (BCG). In some embodiments, the subject has received more than one treatment with BCG. In some embodiments, the subject exhibited recurrence of NMIBC after at least one prior treatment comprising BCG. In some embodiments, the subject has BCG refractory NMIBC. In some embodiments, the subject has relapsed NMIBC. In some embodiments, the subject has or has been diagnosed with one or both of carcinoma in situ (CIS) or high-grade papillary disease. In some embodiments, the subject has or has been diagnosed with T1 bladder cancer, e.g., after at least one prior treatment comprising BCG. In some embodiments, the subject has previously received at least one treatment comprising pembrolizumab, valrubicin, gemcitabine, docetaxel, gemcitabine and mitomycin in combination, gemcitabine and docetaxel in combination, and/or nab-paclitaxel. In some embodiments, the subject receives or has received treatment with an anti-cholinergic agent prior to treatment with the composition.

In some embodiments, the vector is targeted to bladder epithelium tissue. In some embodiments, the non-replicating recombinant adenoviral vector comprises or is a type 5 non-replicating adenoviral vector. In some embodiments, the non-replicating recombinant adenoviral vector is administered at a dose of about 2.25 × 10¹³ viral particles (vp). In some embodiments, the total volume of the dose is about 75 ml. In some embodiments, the non-replicating recombinant adenoviral vector is administered at a dosage of about 1 × 10¹¹ viral particles(vp)/ml to about 3 × 10¹¹ vp/ml.

In some embodiments, the composition is administered or has been administered to a population of subjects. In some embodiments, the method further comprises evaluating or monitoring a complete response (CR) in the population of subjects. In some embodiments, the method further comprises evaluating or monitoring, in the population of subjects, one, two, three, or four of: durability of CR (e.g., median or mean durability of CR), high-grade recurrence-free survival, radical cystectomy free survival, or overall survival. In some embodiments, the population of subjects comprises subjects having or diagnosed with one or both of CIS or high-grade papillary disease.

In some embodiments, about 20-30% (e.g., about 22-26%, e.g., about 24.3%) of the subjects (e.g., subjects with CIS) exhibit high-grade recurrence free survival (RFS) after at least about one year of treatment with the composition. In some embodiments, about 17-27% (e.g., about 20-24%, e.g., about 21.4%) of the subjects (e.g., subjects with CIS) exhibit high-grade recurrence free survival (RFS) after at least about one year (e.g., about 18 months) of treatment with the composition. In some embodiments, about 15-25% (e.g., about 18-22%, e.g., about 19.4%) of the subjects (e.g., subjects with CIS) exhibit high-grade recurrence free survival (RFS) after at least about one year (e.g., about two years) of treatment with the composition.

In some embodiments, durability of CR (e.g., median or mean durability of CR) for the subjects (e.g., subjects with CIS) is about 7-12 months (e.g., about 8-10 months, e.g., about 9.69 months). In some embodiments, about 30-55% (e.g., about 35-50%, e.g., about 40-50%, e.g., about 45.5%) of the subjects (e.g., subjects with CIS) do not exhibit high grade recurrence after at least about one year of treatment with the composition (e.g., after at least about one year following treatment with at least one dose of the composition).

In some embodiments, about 35-55% (e.g., about 40-50%, e.g., about 43.8%) of the subjects (e.g., subjects with papillary disease) exhibit high-grade RFS after at least about one year of treatment with the composition (e.g., after at least about one year following treatment with at least one dose of the composition). In some embodiments, about 28-38% (e.g., about 31-36%, e.g., about 33.3%) of the subjects (e.g., subjects with papillary disease) exhibit high-grade RFS after at least about one year of treatment (e.g., about 18 months) with the composition (e.g., after at least about one year following treatment with at least one dose of the composition). In some embodiments, about 22-32% (e.g., about 25-30%, e.g., about 27.1%) of the subjects (e.g., subjects with papillary disease) exhibit high-grade RFS after at least about one year of treatment (e.g., about two years) with the composition (e.g., after at least about one year following treatment with at least one dose of the composition). In some embodiments, durability of CR (e.g., median or mean durability of CR) in the subjects (e.g., subjects with papillary disease) is about 10-14 months (e.g., about 11-13 months, e.g., about 12.35 months). In some embodiments, about 40-80% (e.g., about 50-70%, e.g., about 60%) of the subjects (e.g., subjects with papillary disease) do not exhibit high grade recurrence after at least about one year of treatment with the composition (e.g., after at least about one year following treatment with at least one dose of the composition).

In some embodiments, durability of CR (e.g., median or mean durability of CR) in the subjects (e.g., subjects with CIS and subjects with papillary disease) is about 5-10 months (e.g., about 6-9 months, e.g., about 7.31 months). In some embodiments, about 20-40% (e.g., about 25-35%, e.g., about 30.5%) of the subjects (e.g., subjects with CIS and subjects with papillary disease) exhibit high-grade RFS after at least about one year of treatment with the composition (e.g., after at least about one year following treatment with at least one dose of the composition). In some embodiments, cystectomy-free survival of the subjects (e.g., subjects with CIS and subjects with papillary disease) is about 50-75% (e.g., about 60-70%, e.g., about 64.5%) after at least about two years of treatment with the composition (e.g., after at least about two years following treatment with at least one dose of the composition).

In some embodiments, overall survival of the subjects (e.g., subjects with CIS) is about 85-95% (e.g., about 91.9%) after at least about two years of treatment with the composition (e.g., after at least about two years following treatment with at least one dose of the composition). In some embodiments, overall survival of the subjects (e.g., subjects with papillary disease) is about 85-95% (e.g., about 93.5%) after at least about two years of treatment with the composition (e.g., after at least about two years following treatment with at least one dose of the composition).

In some embodiments, less than about 2-8% (e.g., about 3-6%, e.g., about 4.9%) of the subjects (e.g., subjects with CIS) exhibit progression to muscle invasion. In some embodiments, less than about 4-8% (e.g., about 5-7%, e.g., about 6.3%) of the subjects (e.g., subjects with papillary disease) exhibit progression to muscle invasion.

In some embodiments, about 15-35% (e.g., about 20-30%, e.g., about 23.3%) of the subjects (e.g., subjects with CIS) exhibit high-grade recurrence free survival (RFS) after at least about 15 months of treatment with the composition (e.g., after at least about 15 months following treatment with at least one dose of the composition). In some embodiments, about 25-50% (e.g., about 30-45%, e.g., about 39.6%) of the subjects (e.g., subjects with papillary disease) exhibit high-grade recurrence free survival (RFS) after at least about 15 months of treatment with the composition (e.g., after at least about 15 months following treatment with at least one dose of the composition).

### BRIEF DESCRIPTION OF THE DRAWING

FIG. 1 shows a graph of durability of response over time for patients with high-grade, BCG unresponsive non-muscle invasive bladder cancer (NMIBC) treated with ADSTILADRIN®). High-grade recurrence-free survival is presented in intervals shown after the month 3 efficacy assessment. CIS = carcinoma in situ; CI = confidence interval; HGRF = high-grade-recurrence-free. The CIS cohort included patients with CIS with and without concomitant papillary disease (Ta/T1). A 95% exact binomial CI for HGRF survival rate was calculated using the Clopper-Pearson method. Median is Kaplan-Meier estimate; the two-sided CI was computed using the method of Brookmeyer and Crowley.

FIG. 2 shows a graph of cystectomy-free survival over time for the same patients in FIG. 1 treated with ADSTILADRIN®. CIS = CIS Only, Ta+CIS, T1+CIS; Papillary Disease = Ta & T1. Cystectomy-free survival is defined as the time from the first dose to the first date of cystectomy or death due to any cause. Cystectomy-free survival rate is the Kaplan-Meier estimate of survivor function at each specific time point. The 95% CI is calculated using the Greenwood’s formula with a log-log transformation.

FIG. 3 shows a graph of overall survival over time for the same patients shown in FIGS. 1 and 2 treated with ADSTILADRIN®. CIS = CIS Only, Ta+CIS, T1+CIS; Papillary Disease = Ta & T1; + = Censored observation.

FIG. 4 shows a graph of incidence of High Grade Recurrence Free Survival (RFS) for the same patients shown in FIGS. 1-3 treated with ADSTILADRIN®. High Grade RFS for patients with CIS or high-grade papillary disease are shown at 3 months, 6 months, 9 months, 12 months, and 15 months.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, or topical. Examples of parental routes include, without limitation, intravesical, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratympanic, intrauterine, intravascular, intravenous (e.g., bolus or drip), intraventricular, and subcutaneous. In some embodiments, administration comprises intravesical administration. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Approximately or About: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cancer: The terms “cancer,” “malignancy,” “neoplasm,” “tumor,” and “carcinoma,” are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a cancer may be characterized by a solid tumor. In some embodiments, a cancer may be characterized by a hematologic tumor. In general, examples of different types of cancers known in the art include, for example, hematopoietic cancers including leukemias, lymphomas (Hodgkin’s and non-Hodgkin’s), myelomas and myeloproliferative disorders; sarcomas, melanomas, adenomas, and carcinomas of solid tissue; squamous cell carcinomas of the mouth, throat, larynx, and lung; liver cancer; genitourinary cancers, such as prostate, cervical, bladder, uterine, endometrial cancer, or renal cell carcinomas; bone cancer; pancreatic cancer; skin cancer; cutaneous or intraocular melanoma; cancer of the endocrine system; cancer of the thyroid gland; cancer of the parathyroid gland; head and neck cancers; breast cancer; gastro-intestinal cancers; nervous system cancers; and benign lesions, such as papillomas. In some embodiments, a cancer comprises or is a bladder cancer, e.g., a high-grade non-muscle-invasive bladder cancer (NMIBC). In some embodiments, a cancer comprises or is carcinoma in situ (CIS) and/or high-grade papillary disease. In some embodiments, a cancer comprises or is Ta or T1 bladder cancer. In some embodiments, a cancer comprises or is a Bacillus Calmette-Guerin (BCG)-resistant cancer.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity). In some embodiments, a combination therapy comprises a composition comprising (i) a non-replicating recombinant adenoviral vector encoding interferon α-2b, and (ii) [N-(3-cholamidopropyl)-N-(3-lactobionamidopropyl)]-cholamide (SYN3), in combination with a checkpoint inhibitor. In some embodiments, a combination therapy comprises a composition comprising (i) a non-replicating recombinant adenoviral vector encoding interferon α-2b, and (ii) [N-(3-cholamidopropyl)-N-(3-lactobionamidopropyl)]-cholamide (SYN3), in combination with an anti-cholinergic agent.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Gene therapy: As used herein, the term “gene therapy” refers to insertion or deletion of specific genomic DNA sequences to treat or prevent a disorder or condition for which such therapy is sought. In some embodiments, the insertion or deletion of genomic DNA sequences occurs in specific cells (e.g., target cells). Target cells may be from a mammal and/or may be cells in a mammalian subject (e.g., a human). In some embodiments, heterologous DNA is transferred to target cells. The heterologous DNA may be introduced into the selected target cells in a manner such that the heterologous DNA is expressed and a therapeutic product encoded thereby is produced. Additionally or alternatively, the heterologous DNA may in some manner mediate expression of DNA that encodes the therapeutic product, or it may encode a product, such as a peptide or RNA that in some manner mediates, directly or indirectly, expression of a therapeutic product. Gene therapy may also be used to deliver a nucleic acid encoding a gene product that replaces a defective gene or supplements a gene product produced by the mammal or the cell in which it is introduced. The heterologous DNA encoding the therapeutic product may be modified prior to introduction into the cells of the afflicted host in order to enhance or otherwise alter the product or expression thereof. Gene therapy may also involve delivery of an inhibitor or repressor or other modulator of gene expression. Gene therapy may include in vivo or ex vivo techniques. In some embodiments, viral and non-viral based gene transfer methods can be used to introduce nucleic acids encoding a polypeptide of interest into mammalian cells or target tissues. Non-viral vector delivery systems include DNA plasmids, naked nucleic acid, and nucleic acid complexed with a delivery vehicle, such as poloxamers or liposomes. Viral vector delivery systems include DNA and RNA viruses, which have either episomal or integrated genomes after delivery to the cell. For a review of gene therapy procedures, see Anderson, Science 256:808-813 (1992); Miller, Nature 357:455-460 (1992); Feuerbach et al., Kidney International 49:1791-1794 (1996); Urnov et al., Nature Reviews Genetics 11, 636-646 (2010); and Collins et al., Proceedings Biologicial Sciences / The Royal Society, 282(1821):pii 20143003 (2015), each of which is hereby incorporated by reference in its entirety.

Inhibitor: As used herein, the term “inhibitor” refers to an agent, condition, or event whose presence, level, degree, type, or form correlates with decreased level or activity of another agent (e.g.., the inhibited agent, or target). In general, an inhibitor may be or include an agent of any chemical class including, for example, small molecules, polypeptides, nucleic acids, carbohydrates, lipids, metals, and/or any other entity, condition, or event that shows the relevant inhibitory activity. In some embodiments, an inhibitor may directly exert its influence upon its target, for example by binding to the target. In some embodiments, an inhibitor may be indirectly exerts its influence by interacting with and/or otherwise altering a regulator of the target, so that level and/or activity of the target is reduced. In some embodiments, an inhibitor comprises or is a checkpoint inhibitor, e.g., a checkpoint inhibitor targeting a checkpoint molecule chosen from PD-1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (e.g., TNFRSF14 or CD270), KIR, A2aR, MHC class I, MHC class II, GAL9, adenosine, or TGF (e.g., TGF beta). In certain embodiments, a checkpoint inhibitor comprises an anti-PD-L1 antibody or a fragment thereof, e.g., Pembrolizumab.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Pharmaceutical composition. As used herein, the term “pharmaceutical composition” refers to a composition in which an active agent (e.g., a non-replicating recombinant adenoviral vector encoding interferon α-2b) is formulated together with one or more pharmaceutically acceptable carriers (e.g., [N-(3-cholamidopropyl)-N-(3-lactobionamidopropyl)]-cholamide (SYN3)). In some embodiments, the active agent is present in unit dose amount appropriate for administration in a therapeutic regimen that shows a statistically significant probability of achieving a predetermined therapeutic effect when administered to a relevant population. A pharmaceutical composition may be specially formulated for administration in solid or liquid form, including those adapted for the following: oral administration, for example, drenches (aqueous or non-aqueous solutions or suspensions), tablets (e.g., targeted for buccal, sublingual, and systemic absorption), boluses, powders, granules, pastes for application to the tongue; parenteral administration, for example, by intravesical, subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In certain embodiments, a pharmaceutical composition is formulated as a suspension (e.g., sterile suspension) for intravesical instillation. In some embodiments, a pharmaceutical composition is intended and suitable for administration to a human subject. In some embodiments, a pharmaceutical composition is sterile and substantially pyrogen-free.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; pH buffered solutions; polyesters, polycarbonates and/or polyanhydrides; and other non-toxic compatible substances employed in pharmaceutical formulations. In some embodiments, a pharmaceutically acceptable carrier comprises [N-(3-cholamidopropyl)-N-(3-lactobionamidopropyl)]-cholamide (SYN3).

Prevent or Prevention: The term “prevent” or “prevention,” as used herein, refers to a delay of onset, and/or reduction in frequency and/or severity of one or more symptoms of a particular disease, disorder or condition (e.g., a cancer described herein, such as bladder cancer (e.g., high-grade NMIBC)). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. Prevention may be considered complete when onset of a disease, disorder or condition has been delayed for a predefined period of time.

Refractory: As used herein, the term “refractory” refers to any subject that does not respond with an expected clinical efficacy following the administration of provided compositions as normally observed by practicing medical personnel.

Response: As used herein, a response to treatment may refer to any beneficial alteration in a subject’s condition that occurs as a result of or correlates with treatment. Such alteration may include stabilization of the condition (e.g., prevention of deterioration that would have taken place in the absence of the treatment), amelioration of one or more symptoms of the condition, and/or improvement in the prospects for cure of the condition. It may refer to a subject’s response or to a tumor’s response. Tumor or subject response may be measured according to a wide variety of criteria, including clinical criteria and objective criteria. Techniques for assessing response include, but are not limited to, presence or level of tumor markers in a sample obtained from a subject (e.g., in a biopsy), cytology, cystoscopy, clinical examination, positron emission tomography, chest X-ray CT scan, MRI, ultrasound, endoscopy, laparoscopy, and/or histology. Many of these techniques can be used to determine the size of a tumor or otherwise determine the total tumor burden. Exact response criteria can be selected in any appropriate manner, provided that when comparing groups of tumors and/or patients, groups to be compared are assessed based on same or comparable criteria for determining response rate. One of ordinary skill in the art will be able to select appropriate criteria.

Sample: As used herein, the term “sample” refers to a biological sample obtained or derived from a source of interest (e.g., a tumor), as described herein. In some embodiments, a biological sample comprises biological tissue or fluid. In some embodiments, a biological sample may comprise a tissue biopsy sample; bone marrow; blood; blood cells; ascites; tissue or fine needle biopsy samples; cell-containing body fluids; free-floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages, such as a ductal lavages or broncheoalveolar lavages; aspirates; scrapings; bone marrow specimens; surgical specimens; other body fluids, secretions, and/or excretions; and/or cells therefrom. In some embodiments, a biological sample comprises cells obtained from an individual, e.g., from a mammal (e.g., a human). In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or polypeptides extracted from a sample or obtained by subjecting a primary sample to techniques, such as amplification or reverse transcription of mRNA, isolation, and/or purification of certain components.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein (e.g., a bladder cancer or tumor, e.g. high-grade non-muscle-invasive bladder cancer (NMIBC)). In some embodiments, a subject is susceptible to a disease, disorder, or condition. In some embodiments, a susceptible subject is predisposed to and/or shows an increased risk (as compared to the average risk observed in a reference subject or population) of developing the disease, disorder, or condition. In some embodiments, a subject has been diagnosed with one or more diseases, disorders or conditions. In some embodiments, a subject displays one or more symptoms of a disease, disorder or condition. In some embodiments, a subject does not display a particular symptom (e.g,. clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered. In some embodiments, a subject is receiving or has received certain therapy to diagnose and/or to treat a disease, disorder, or condition. In some embodiments, a subject is part of a population of subjects, e.g., a population of patients undergoing a Clinical Trial of a composition described herein.

Therapeutically effective amount: As used herein, the term “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, or urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

The present disclosure is based, in part, on the discovery that administration of compositions (e.g., pharmaceutical compositions) described herein are effective to treat a cancer (e.g., a bladder cancer, such as high-grade non-muscle-invasive bladder cancer (NMIBC)) for a relatively long-term duration, e.g., for at least one year of administration. In some embodiments, a composition comprises: (i) a non-replicating recombinant adenoviral vector encoding interferon α-2b, and (ii) [N-(3-cholamidopropyl)-N-(3-lactobionamidopropyl)]-cholamide (SYN3). In some embodiments, the composition is administered as a monotherapy. In other embodiments, the composition is administered in combination with one or more other therapeutic agents (e.g., a checkpoint inhibitor and/or an anti-cholinergic agent).

### Subjects or Patient Population

The present disclosure includes, among other things, methods of treating a subject or patient population for cancer (e.g., a bladder cancer, such as high-grade non-muscle-invasive bladder cancer (NMIBC)). The disclosure also provides methods of monitoring or evaluating efficacy of a composition (e.g., described herein) administered to a subject or patient population. In some embodiments, a subject or patient population comprises or has a bladder cancer, e.g., a high-grade NMIBC. The disclosure includes, in part, treatment of a subject using a composition described herein, such that growth of cancerous tumors (e.g., bladder tumors, e.g., high-grade NMIBC tumors) is inhibited or reduced.

In some embodiments, a subject is a human, e.g., a human patient having a disorder or condition characterized by abnormal cellular proliferation, e.g., of bladder tissue. In some embodiments, a subject is a “non-human animal,” such as a non-human primate. In some embodiments, the subject is a human. In some embodiments, the subject is a human patient in need of enhancement of an immune response. The methods and compositions described herein are suitable for treating human patients having a disorder that can be treated by promoting anti-tumor effects, such as one, two, three, or four of anti-proliferation, apoptosis, angiogenesis inhibition, and/or immune augmentation.

In some embodiments, a subject or patient population comprises or has carcinoma in situ (CIS) and/or high-grade papillary disease. In some embodiments, a subject or patient population comprises or has Ta or T1 bladder cancer. In some embodiments, a subject or patient population comprises or has a Bacillus Calmette-Guerin (BCG)-resistant cancer. In some embodiments, a subject comprises or has a BCG-refractory or a relapsed cancer (e.g., relapsed NMIBC). In some embodiments, a subject has BCG-unresponsive NMIBC. In some embodiments, a subject deemed BCG resistant, refractory or unresponsive may have received a full or partial dose or course of BCG, e.g., as compared to an approved or standard of care regimen (e.g., a regimen on regulatory label instructions). In some embodiments, a subject may have received a partial dose because a subject was unable or unwilling to receive a full dose or course of BCG and/or because insufficient BCG was available for administration to a subject. In some embodiments, a subject or patient population comprises or has an Eastern Cooperative Oncology Group status of 2 or less than 2 (see, e.g., Oken et al., Am J. Clin. Oncol. 5:649-655 (1982)).

### Methods of Treatment

The present disclosure includes, among other things, methods of treating cancer (e.g., a bladder cancer, such as high-grade non-muscle-invasive bladder cancer (NMIBC)) in a subject or patient population by administering a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)). In some embodiments, the disclosure provides a method of inhibiting growth of tumor cells (e.g., bladder tumor cells, e.g., high-grade NMIBC tumor cells) in a subject, comprising administering a therapeutically effective amount of a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) to a subject or patient population described herein, e.g., in accordance with a dosage regimen described herein.

Compositions described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) can be administered according to a dosage regimen described herein to treat (e.g., inhibit, reduce, ameliorate, or prevent) a cancer (e.g., a bladder cancer, e.g., high-grade NMIBC) in a subject or a patient population described herein.

Compositions described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) may be used as a monotherapy to inhibit the growth of cancerous tumors in a subject or patient population described herein. Alternatively, compositions described herein may be used in combination with other therapeutic agents, procedures, or modalities to treat or prevent cancerous disorders (e.g., bladder cancer, e.g., high-grade NMIBC).

### Dosage Regimens

In some embodiments, a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) is administered at a dose of about 2.25 × 10¹³ viral particles (vp), e.g., in a total volume of about 10 ml, about 25 ml, about 50 ml, about 75 ml, about 100 ml, about 150 ml, or about 200 ml every 3 months (e.g., at 3 months, at 6 months, at 9 months, at 12 months, at 15 months, at 18 months, at 21 months, at 24 months, at 36 months, at 48 months, at 60 months, or longer) after initial treatment with a composition described herein. In some embodiments, the dose is administered by intravesical instillation.

In some embodiments, a composition described herein is administered at a dose of about 1.0 × 10¹¹ vp/ml to about 3.0 × 10¹¹ vp/ml (e.g., about 1 × 10¹¹ vp/ml, 1.2 × 10¹¹ vp/ml, 1.4 × 10¹¹ vp/ml, 1.6 × 10¹¹ vp/ml, 1.8 × 10¹¹ vp/ml, 2.0 × 10¹¹ vp/ml, 2.2 × 10¹¹ vp/ml, 2.4 × 10¹¹ vp/ml, 2.6 × 10¹¹ vp/ml, 2.8 × 10¹¹ vp/ml, or about 3.0 × 10¹¹ vp/ml) every 3 months (e.g., at 3 months, at 6 months, at 9 months, at 12 months, at 15 months, at 18 months, at 21 months, at 24 months, and longer after initial treatment with a composition described herein). In certain embodiments, a composition described herein is administered at a dose of about 1.0 × 10¹¹ vp/ml. In certain embodiments, a composition described herein is administered at a dose of about 3.0 × 10¹¹ vp/ml.

### Combination Therapies

The present disclosure includes, among other things, compositions described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) for use in combination with at least one other therapeutic agents, procedures, or modalities. In some embodiments, other therapeutic agents, procedures, or modalities comprise one, two, three, four, or five of: a standard of care treatment for a cancer (e.g., a bladder cancer, e.g., high-grade NMIBC); an antibody or antigen-binding fragment thereof; an immunomodulator (e.g., an activator of a costimulatory molecule or an inhibitor of an inhibitory molecule); a vaccine (e.g., a therapeutic cancer vaccine); or any other form of immunotherapy described herein to treat and/or inhibit the growth of cancerous tumors in a subject or patient population described herein.

In some embodiments, one or more therapeutic agents, procedures, or modalities (e.g., described herein) is administered prior to, concurrently, and/or after treatment with a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)). A composition described herein and a therapeutic agent, procedure, or modality can be administered or used simultaneously or sequentially in any order. Any combination and sequence of a composition described herein and a therapeutic agent, procedure, or modality can be used.

In some embodiments, combined administration of a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) and an additional therapeutic agent results in an improvement in cancer to an extent that is greater than one produced by either the composition or the additional therapeutic agent alone. The difference between the combined effect and the effect of each agent alone can be a statistically significant difference. In some embodiments, the combined effect can be a synergistic effect. In some embodiments, combined administration of a composition described herein and an additional therapeutic agent allows administration of the additional therapeutic agent at a reduced dose, at a reduced number of doses, and/or at a reduced frequency of dosage compared to a standard dosing regimen, e.g., an approved dosing regimen for the additional therapeutic agent.

In some embodiments, treatment methods described herein are performed on subjects for whom other treatments of the medical condition (e.g., comprising Bacillus Calmette-Guerin (BCG)) have failed or have had less success in treatment through other means. Additionally, the treatment methods described herein can be performed in conjunction with one or more additional treatments of the medical condition. For instance, the method can comprise administering a cancer regimen, e.g., nonmyeloablative chemotherapy, surgery, hormone therapy, and/or radiation, prior to, substantially simultaneously with, or after the administration of a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)).

In some embodiments, at least one anti-cholinergic agent is administered prior to, concurrently, or after treatment with a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)). In some embodiments, an anti-cholinergic agent prevents, treats, or relieves urinary urgency during treatment with a composition described herein. In some embodiments at least one anti-cholinergic agent is administered prior to treatment with a composition described herein, e.g., at least about 5 minutes, about 10 minutes, about 20 minutes, about 30 minutes, about 1 hour, about 2 hours, about 3 hours, about 4 hours, about 5 hours or more, before administration of a composition described herein.

An anti-cholinergic can include, but is not limited to, ipratropium, atropine, glycopyrronium, or tiotropium, or salts, solvates, esters, isomers, polymorphs, or derivatives of any of the foregoing. In some embodiments, an anti-cholinergic agent comprises trospium chloride (e.g., as described in U.S. Pat. No. 5,998,430, which is hereby incorporated by reference in its entirety). In some embodiments, an anti-cholinergic agent comprises tiotropium bromide (e.g., as described in WO 2004/064789, which is hereby incorporated by reference in its entirety). In some embodiments, an anti-cholinergic agent comprises glycopyrronium bromide (e.g., as described in WO 2001/17480, which is hereby incorporated by reference in its entirety).

In some embodiments, one or more additional therapeutic agents comprises or is a biological agent or an immunologic agent. In some embodiments, a biological agent comprises tumor-infiltrating lymphocytes, CAR T-cells, antibodies, antigens, therapeutic vaccines (e.g., made from a patient’s own tumor cells or other substances, such as antigens produced by certain tumors (e.g., bladder tumors)), immune-modulating agents (e.g., cytokines, e.g., immunomodulatory drugs or biological response modifiers), checkpoint inhibitors, or other immunologic agents. In certain embodiments, immunologic agents include immunoglobins, immunostimulants (e.g., bacterial vaccines, colony stimulating factors, interferons, interleukins, therapeutic vaccines, vaccine combinations, or viral vaccines) and/or immunosuppressive agents (e.g., calcineurin inhibitors, interleukin inhibitors, or TNF alpha inhibitors).

Additional therapeutic agents for combination therapies described herein include immune checkpoint therapeutics (e.g., pembrolizumab, nivolumab, ipilimumab, atezolizumab, avelumab, durvalumab, tremelimumab, or cemiplimab), other monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, nimotuzumab, lambrolizumab, pidilizumab, siltuximab, BMS-936559, RG7446/MPDL3280A, or MEDI4736), antibody-drug conjugates (e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. 1045:1-27 (2013); Bouchard et al., Bioorganic Med. Chem. Lett. 24: 5357-5363 (2014), which are each hereby incorporated by reference in their entirety), or any combination thereof.

In some embodiments, a checkpoint inhibitor is administered to a patient (e.g., a patient having bladder cancer (e.g., NMIBC)) in combination with a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)). In some embodiments, a checkpoint inhibitor is administered if a patient is determined to be positive for a tumor (e.g., a high-grade tumor, e.g., a high-grade bladder tumor), e.g., as determined using one or more assays described herein (e.g., a biopsy). In some embodiments, a checkpoint inhibitor targets a checkpoint molecule chosen from PD-1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (e.g., TNFRSF14 or CD270), KIR, A2aR, MHC class I, MHC class II, GAL9, adenosine, or TGF (e.g., TGF beta).

In some embodiments, a checkpoint inhibitor comprises an anti-PD-1 antibody or a fragment thereof. In some embodiments, an anti-PD-1 antibody comprises or is chosen from Pembrolizumab, Nivolumab, or Pidilizumab. In some embodiments, an anti-PD-1 antibody compirises or is Pembrolizumab. Pembrolizumab (known as Lambrolizumab, MK-3475, MK03475, SCH-900475, or KEYTRUDA®) is a humanized IgG4 monoclonal antibody that binds to PD-1 (e.g., as disclosed in Hamid, O. et al. (2013) New England Journal of Medicine 369 (2): 134-44, WO2009/114335, and U.S. Pat. No. 8,354,509, each of which is hereby incorporated by reference in its entirety). Amino acid sequences of a heavy chain sequence and light chain sequence of Pembrolizumab are provided in Table 1.

TABLE 1

Heavy chain and light chain amino acid sequences of Pembrolizumab

| | | |
|---|---|---|
| Pembrolizumab | | |
| SEQ ID NO: 1 | Heavy chain | QVQLVQSGVEVKKPGASVKVSCKASGYTFTNYYMYWVRQAPGQGLEWM GGINPSNGGTNFNEKFKNRVTLTTDSSTTTAYMELKSLQFDDTAVYYCARR DYRFDMGFDYWGQGTTVTVSSASTKGPSVFPLAPCSRSTSESTAALGCLVK DYFPEPVTVSWNSGALTSGVHTFPAVLQSSGLYSLSSVVTVPSSSLGTKTYT CNVDHKPSNTKVDKRVESKYGPPCPPCPAPEFLGGPSVFLFPPKPKDTLMISR TPEVTCVWDVSQEDPEVQFNWYVDGVEVHNAKTKPREEQFNSTYRVVSV LTVLHQDWLNGKEYKCKVSNKGLPSSIEKTISKAKGQPREPQVYTLPPSQEE MTKNQVSLTCLVKGFYFSDIAVEWESNGQPENNYKTTPPVLDSDGSFFLYS RLTVDKSRWQEGNVFSCSVMHEALHNHYTQKSLSLSLGK |
| SEQ ID NO: 2 | Light chain | EIVLTQSPATLSLSPGERATLSCRASKGVSTSGYSYLHWYQQKPGQAPRLLI YLASYLESGVPARFSGSGSGTDFTLTISSLEPEDFAVYYCQHSRDLPLTFGGG TKVEIKRTVAAPSVFIFPPSDEQLKSGTASVVCLLNNFYPREAKVQWKVDNA LQSGNSQESVTEQDSKDSTYSLSSTLTLSKADYEKHKVYACEVTHQGLSSPV TKSFNRGEC |

### Methods of Evaluating or Monitoring Efficacy

The present disclosure includes, among other things, methods of evaluating or monitoring efficacy of a composition described herein in a subject (e.g., a subject having a cancer, e.g., a cancer described herein, e.g., high-grade non-muscle-invasive bladder cancer (NMIBC). Assays and techniques for evaluating efficacy include, but are not limited to, presence or level of tumor markers in a sample obtained from a subject (e.g., in a biopsy), cytology, cystoscopy, clinical examination, positron emission tomography, MRI, ultrasound, endoscopy, laparoscopy, and/or histology. In some embodiments, one or more assays are performed about 1 week, 2 weeks, 3 weeks, 1 month, 2 months, 3 months, 4 months, 5 months, 6 months, 7 months, 8 months, 9 months, 10 months, 11 months, 12 months, 15 month, 18 months, 21 months, 24 months, or longer, following administration of a dose of a composition described herein.

In some embodiments, an assay is performed on a subject or a sample from a subject. In some embodiments, an assay is performed on a patient population or one or more samples from a patient population. In some embodiments, a sample is acquired from a subject. In some embodiments, a sample comprises a blood sample (e.g., a plasma or serum sample), saliva sample, tissue sample, or urine sample. In some embodiments, an assay comprises acquiring a biopsy sample (e.g., of bladder tissue) from the subject, e.g., a biopsy from a subject after at least one year of treatment with the composition. In some embodiments, a biopsy is performed in addition to one or both of cytology of a sample (e.g., a urine sample) and/or cystoscopy of a subject. In other embodiments, a biopsy is performed instead of one or both of cytology of a sample (e.g., a urine sample) and/or cystoscopy of a subject. In some embodiments, a biopsy sample comprises a bladder tissue sample. In some embodiments, prior to administration of a composition described herein, a biopsy sample indicates a patient is positive for a tumor (e.g., a high-grade tumor, e.g., a high-grade bladder tumor). In some embodiments, a checkpoint inhibitor (e.g., as described herein) is administered if a biopsy from a patient is determined to be tumor positive.

In some embodiments, a subject administered a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) is evaluated or monitored for one or both of biodistribution or shedding of a vector in the composition. In some embodiments, a sample comprises a blood sample (e.g., a plasma or serum sample), saliva sample, tissue sample, or urine sample. In some embodiments, a vector described herein (e.g., a non-replicating recombinant adenoviral vector encoding interferon α-2b) in the composition is detectable in the sample (e.g., a blood or urine sample). In other embodiments, a vector described herein is not detectable in the sample (e.g., a blood or urine sample), e.g., after at least 1 week, 2 weeks, 3 weeks, 1 month, 2 months, 3 months, 6 months, or at longer administering the composition in a dosage regimen described herein.

In some embodiments, a subject, treated with a composition described herein, exhibits a complete response (CR) to treatment with the composition. A “complete response subject,” as used herein, refers to a subject having a cancer (e.g., a cancer described herein, e.g., high-grade non-muscle-invasive bladder cancer (NMIBC)) who exhibits a complete response (e.g., a complete remission) to treatment with a composition described herein. In some embodiments, a subject, treated with a composition described herein, exhibits a partial response to treatment with the composition. A “partial response subject,” as used herein, refers to a subject having a cancer (e.g., a cancer described herein, e.g., high-grade non-muscle-invasive bladder cancer (NMIBC)) who exhibits a partial response to treatment with a composition described herein. In some embodiments, a subject, treated with a composition described herein, exhibits a non-response to treatment with the composition. A “non-response subject,” as used herein, refers to a subject having a cancer (e.g., a cancer described herein, e.g., high-grade non-muscle-invasive bladder cancer (NMIBC)) who does not exhibit a response to treatment with a composition described herein.

A complete response, partial response, or non-response in a subject or a population of subjects can be identified by an assay described herein, e.g., by one, two, or three of cytology (e.g., urine cytology), cystoscopy, or biopsy for a tumor (e.g., of bladder tissue). In some embodiments, a subject with a CR is negative for a tumor or tumor type (e.g., a high-grade tumor, e.g., a high-grade bladder tumor) as determined by an assay described herein, e.g., one, two, or three of negative urine cytology, lesion-free cystoscopy, or negative biopsy for a tumor (e.g., of bladder tissue). In some embodiments, a population of subjects, treated with a composition described herein, exhibits one, two, or three of a complete response (CR), a partial response, or a non-response to treatment with the composition.

In some embodiments, a population of subjects (e.g., subjects with CIS), treated with a composition described herein, exhibits about 10%, about 13%, about 15%, about 17%, about 20%, about 21%, about 22%, about 23%, about 24%, about 25%, about 26%, about 27%, about 28%, about 29%, about 30%, about 35%, about 37%, about 40%, about 42%, about 45%, about 50% or more durable response or high-grade recurrence free survival (RFS) after at least about one year of treatment with the composition (e.g., after at least about 13 months, about 14 months, about 15 months, about 16 months, about 17 months, about 18 months, about 19 months, about 20 months, about 21 months, about 22 months, about 23 months, about 2 years or longer of treatment with the composition).

In some embodiments, a population of subjects (e.g., patients with papillary disease, such as patients with Ta or T1 disease), treated with a composition described herein, exhibits about 20%, about 21%, about 22%, about 23%, about 24%, about 25%, about 26%, about 27%, about 28%, about 29%, about 30%, about 35%, about 37%, about 40%, about 42%, about 43%, about 44%, about 45%,, about 46%, about 47%, about 48%, about 49, about 50%, about 52%, about 53%, about 55%, about 57%, about 60%, about 62%, about 65%, about 67%, about 70%, or more durable response or high-grade RFS after at least about one year of treatment with the composition (e.g., after at least about 13 months, about 14 months, about 15 months, about 16 months, about 17 months, about 18 months, about 19 months, about 20 months, about 21 months, about 22 months, about 23 months, about 2 years or longer of treatment with the composition). In some embodiments, durability of CR (e.g., median durability of CR, mean durability of CR) for a population of subject (e.g., subjects with CIS or papillary disease), treated with a composition described herein, is about 1 month, about 2 months, about 3 months, about 4 months, about 5 months, about 6 months, about 7 months, about 8 months, about 9 months, about 10 months, about 11 months, about 12 months or longer (e.g., after at least about 13 months, about 14 months, about 15 months, about 16 months, about 17 months, about 18 months, about 19 months, about 20 months, about 21 months, about 22 months, about 23 months, about 2 years or longer of treatment with the composition).

In some embodiments, a population of subjects (e.g., subjects with CIS or papillary disease), treated with a composition described herein, exhibits less than a 5% decrease (e.g., about 4%, about 3%, about 2%, about 1%, or less) in durable response or high-grade RFS from 12 months of treatment with a composition described herein to 15 months or longer of treatment with a composition described herein (e.g., about 16 months, about 17 months, about 18 months, about 19 months, about 20 months, about 21 months, about 22 months, about 23 months, about 2 years or longer of treatment with the composition).

In some embodiments, after treatment of a subject (or population of subjects) with a composition described herein, a subject’s results from one or more assays described herein can be compared to a corresponding control result (e.g., results from a subject (or a population of subjects) free of disease/disorder, and/or results from the same subject (or population of subjects) at any time before administration of a dose of a composition described herein). In some embodiments, after the comparison, a determination can be made about treatment or administration. For example, a determination can be made that treatment should continue or cease, that the dosage should be increased or decreased, and/or that the dosing regimen should be altered. In some embodiments, a determination is made to switch from one therapy to another, e.g., to switch from monotherapy treatment with a composition described herein to a combination therapy described herein.

In some embodiments, a subject (or population of subjects) no longer requires or is able to avoid a cystectomy after treatment with a composition described herein, e.g., after treatment for at least about 1 month, about 2 months, about 3 months, about 4 months, about 5 months, about 6 months, about 7 months, about 8 months, about 9 months, about 10 months, about 11 months, about 12 months or longer (e.g., at least about 13 months, about 14 months, about 15 months, about 16 months, about 17 months, about 18 months, about 19 months, about 20 months, about 21 months, about 22 months, about 23 months, about 2 years or longer).

Methods described herein can include preparing and/or providing a report, such as in electronic, web-based, or paper form. The report can include one or more outputs from a method described herein, e.g., a subject’s response to a treatment described herein. In some embodiments, a report is generated, such as in paper or electronic form, which identifies one or more results and/or endpoints described herein for a subject, and optionally, a recommended course of therapy. In some embodiments, the report includes an identifier for the subject. In one embodiment, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a subject, e.g., identified in the report. For example, the report can include information, or a recommendation, on the administration of a composition described herein and/or one or more additional therapeutics to the subject. The report can be delivered, e.g., to an entity described herein, within 7, 14, 21, 30, or 45 days from performing a method described herein.

In some embodiments, a report is generated to memorialize each time a subject is assessed using a method described herein. The subject can be reevaluated at intervals, such as every month, every two months, every six months or every year, or more or less frequently, to monitor the subject for responsiveness to a composition described herein and/or for an improvement in one or more cancer symptoms, e.g., described herein. In some embodiments, the report can record at least the treatment history of the subject.

In one embodiment, the method further includes providing a report to another party. The other party can be, for example, the subject, a caregiver, a physician, an oncologist, a hospital, clinic, third-party payor, insurance company or a government office.

### Compositions and Administration

The present disclosure includes, among other things, compositions comprising: (i) a recombinant non-replicating adenoviral vector encoding interferon, e.g., interferon α-2b, and (ii) [N-(3-cholamidopropyl)-N-(3-lactobionamidopropyl)]-cholamide (SYN3). In some embodiments, the composition comprises nadofaragene firadenovac, which is a replication-deficient adenovirus type 5 (Ad5) vector encoding the human interferon alpha 2 (IFNA2, interferon alpha-2b) gene under the control of the cytomegalovirus (CMV) immediate-early enhancer/promoter. A composition described herein can be incorporated into a pharmaceutical composition. Such a pharmaceutical composition can be useful, e.g., for the prevention and/or treatment of diseases, e.g., cancer (e.g., bladder cancer, e.g., NMIBC). In some embodiments, a pharmaceutical composition can be formulated to include a pharmaceutically acceptable carrier or excipient.

Compositions described herein can include or encode type 1 and/or type 2 interferons, including deletion, insertion, or substitution variants thereof, biologically active fragments, and allelic forms. Type 1 interferons include interferon-α, -β, -ε, -κ, -ω, -δ, -ζ and -τ and their subtypes, while Type 2 interferons are referred to as interferon-y (see, e.g., Lee et al., Front. Immunol. 9:2061 (2018)). Particular interferon-α′s include human interferon α subtypes including, but not limited to, α-1 (GenBank Accession Number NP 076918), α-1b (GenBank Accession Number AAL35223), α-2, α-2a (GenBank Accession Number NP000596), α-2b (GenBank Accession Number AAP20099), α-4 (GenBank Accession Number NP066546), α-4b (GenBank Accession Number CAA26701), α-5 (GenBank Accession Numbers NP 002160 and CAA26702), α-6 (GenBank Accession Number CAA26704), α-7 (GenBank Accession Numbers NP 066401 and CAA 26706), α-8 (GenBank Accession Numbers NP002161 and CAA 26903), α-10 (GenBank Accession Number NP 002162), α-13 (GenBank Accession Numbers NP 008831 and CAA 53538), α-14 (GenBank Accession Numbers NP 002163 and CAA 26705), α-16 (GenBank Accession Numbers NP 002164 and CAA 26703), α-17 (GenBank Accession Number NP 067091), α-21 (GenBank Accession Numbers P01568 and NP002166), and consensus interferons as described in U.S. Pat. No. 5,541,293; U.S. Pat. No. 4,897,471; and U.S. Pat. No. 4,695,629; and hybrid interferons as described in U.S. Pat. No. 4,414,150. Interferon-y’s are described in, e.g., EP 77,670A and EP 146,354A, and GenBank Accession Number NP 002168. Particular compositions of the disclosure comprise a recombinant adenoviral vector encoding an interferon-α described in U.S. Pat. No. 6,835,557, e.g., with or without a signal sequence. In some embodiments, a non-replicating recombinant adenoviral vector comprises or is a type 5 non-replicating adenoviral vector. In some embodiments, a non-replicating recombinant adenoviral vector is a recombinant adenoviral vector described in, e.g., U.S. Pat. No. 6,210,939. In some embodiments, a recombinant adenoviral vector encodes at least one IFN α-2 (e.g., one or both of IFN α-2a or IFN α-2b). In certain embodiments, a recombinant adenoviral vector encodes human IFN α-2b. In some embodiments, a pharmaceutical composition described herein comprises an adenovirus vector-based gene therapy expressing interferon IFN-α2b.

In some embodiments, a recombinant adenoviral vector comprises a non-replicating adenoviral vector. In some embodiments, a recombinant adenoviral vector comprises a replication-competent adenoviral vector. In some embodiments, administration of a recombinant adenoviral vector described herein deliver a copy of a gene for IFN-α2b to cells (e.g., urothelial cells) in a subject. Production of IFN-α2b in a subject may result in one or more anti-tumor effects comprising one, two, three, or four of anti-proliferation, apoptosis, angiogenesis inhibition, and immune augmentation. Administration of a composition comprising a recombinant adenoviral vector described herein can increase a level and/or a duration of exposure of a tumor (e.g., of bladder tissue) to IFN-α2b. Transduction of urothelial cells with a composition comprising a recombinant adenoviral vector described herein can result in sustained over-expression of IFN-α2b, e.g., resulting in anti-tumor effects.

In some embodiments, a composition described herein (e.g., a composition comprising: i) a non-replicating recombinant adenoviral vector encoding interferon α-2b, and (ii) [N-(3-cholamidopropyl)-N-(3-lactobionamidopropyl)]-cholamide (SYN3)) can be formulated as a sterile formulation for injection in accordance with conventional pharmaceutical practices. In some embodiments, a composition described herein is a sterile suspension formulation for intravesical instillation. In some embodiments, a composition described herein comprises a polyamide surfactant (e.g., SYN3) to enhance transduction of an adenoviral vector described herein in urothelium tissue of a subject.

A suitable dose of a composition described herein, which dose is capable of treating or preventing a disorder in a subject (e.g., a cancer, e.g., bladder cancer, e.g., NMIBC), can depend on a variety of factors including, e.g., the age, sex, and weight of a subject to be treated and the particular inhibitor compound used. For example, a different dose of one composition including a recombinant adenoviral vector described herein may be required to treat a subject with a cancer (e.g., NMIBC) as compared to the dose of a different formulation of that antibody. Other factors affecting the dose administered to the subject include, e.g., the type or severity of the disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors, such as the disease state, age, sex, and weight of an individual, and ability of a composition described herein to elicit a desired response in the individual, e.g., amelioration of at least one condition parameter, e.g., amelioration of at least one symptom of a cancer (e.g., bladder cancer, e.g., NMIBC) as described herein. For example, a therapeutically effective amount of a composition described herein can inhibit (lessen the severity of or eliminate the occurrence of) and/or prevent a particular disorder, and/or any one of the symptoms of a particular disorder known in the art or described herein. A therapeutically effective amount is also one in which any toxic or detrimental effects of a composition are outweighed by therapeutically beneficial effects.

In some embodiments, a composition described herein is formulated for dosing at 2.25 × 10¹³ viral particles (vp) in a total volume of about 75 ml, e.g., for intravesical instillation every three months. In some embodiments, a single dose of a composition described herein can comprise 4 vials, each of which comprises 3.0 × 10¹¹ vp/mL of a non-replicating recombinant adenoviral vector and excipients comprising [[N-(3-cholamidopropyl)-N-(3-lactobionamidopropyl)]-cholamide (Syn3) (e.g., 20 mg of Syn3), citric acid monohydrate (e.g., 0.2 mg of citric acid monohydrate), sodium citrate dihydrate (e.g., 0.8 mg of sodium citrate dehydrate), polysorbate 80 (Tween 80) (e.g., 10 mg of Tween 80), hydroxypropyl-beta-cyclodextrin (e.g., 164 mg of hydroxypropyl-beta-cyclodextrin), sodium dihydrogen phosphate dihydrate (e.g., 28 mg of sodium dihydrogen phosphate dihydrate), tromethamine (e.g., 28 mg of tromethamine), sucrose (e.g., 334 mg of sucrose), magnesium chloride hexahydrate (e.g., 6 mg of magnesium chloride hexahydrate), glycerol (e.g., 1674 mg of glycerol), and water (e.g., in a 20 ml extractable volume).

In some embodiments, a pharmaceutical composition described herein is substantially free of contaminants (e.g., components (e.g., DNA and protein) of host cells (e.g., HEK293 cells) and/or serum (e.g., fetal bovine serum)). In some embodiments, a pharmaceutical composition described herein comprises trace amounts of contaminants (e.g., components (e.g., DNA and protein) of host cells (e.g., HEK293 cells) and/or serum (e.g., fetal bovine serum)). In some embodiments, a pharmaceutical composition described herein is substantially free of preservative.

Selection or use of any particular form may depend, in part, on the intended mode of administration and therapeutic application. For example, compositions intended for systemic or local delivery can be in the form of injectable or infusible solutions. Accordingly, compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravesical, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion. Administration can be systemic or local. Route of administration can be parenteral, for example, administration by intravesical instillation or injection. In some embodiments, intravesical administration can be accomplished by means of a device, such as a catheter. In some embodiments, about 75 ml of a composition described herein is administered into a bladder of a subject by a urinary catheter and left in the bladder for about 1 hour as the subject is repositioned to maximize bladder surface exposure to the composition (e.g., about every 15 minutes).

In some embodiments, a composition described herein can be formulated for storage at a temperature below 0° C. (e.g., about -20° C. or about -80° C.). In some embodiments, the composition can be formulated for storage for up to 2 years (e.g., one month, two months, three months, four months, five months, six months, seven months, eight months, nine months, 10 months, 11 months, 1 year, or 2 years) at about 2° C. to about 8° C. (e.g., 4° C.). In some embodiments, a composition described herein is thawed at room temperature (e.g., about 20° C. to about 25° C.) until liquid prior to administration to a subject described herein.

Those of skill in the art will appreciate that data obtained from cell culture assays and animal studies can be used in formulating a range of dosage for use in humans. Appropriate dosages of compositions described herein lie generally within a range of circulating concentrations of the compositions that include the ED₅₀ with little or no toxicity. A dosage may vary within this range depending upon the dosage form employed and route of administration utilized. For a composition described herein, a therapeutically effective dose can be estimated initially from cell culture assays. A dose can be formulated in animal models to achieve a circulating plasma concentration range that includes an IC₅₀ as determined in cell culture. Such information can be used to more accurately determine useful doses in humans. Levels in plasma may be measured, for example, by high performance liquid chromatography. In some embodiments, e.g., where local administration (e.g., to bladder tissue) is desired, cell culture or animal modeling can be used to determine a dose required to achieve a therapeutically effective concentration within a local site.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. The example is provided for illustrative purposes only. It is not to be construed as limiting the scope or content of the disclosure in any way.

### EXAMPLES

### Example 1. Clinical Study of Intravesical Instillation of an Adenovirus Vector-Based Gene Therapy ADSTILADRIN® (Nadofaragene Radenovec)

The safety and efficacy of a drug formulation of recombinant adenovirus (rAd)-IFNα and Syn3 (Nadofaragene firadenovac (ADSTILADRIN®)) was evaluated in a multicenter, open-label single arm clinical study in patients with BCG-unresponsive, high-grade non-muscle invasive bladder cancer, where the definition “BCG Unresponsive” was patients with high-grade, non-muscle invasive bladder cancer who had been treated with adequate BCG and were unlikely to benefit from and should not receive further intravesical BCG (Study 2, NCT02773849). Patients with current or previous evidence of muscle invasive (muscularis propria) or metastatic disease were excluded from the study.

Eligible patients were aged 18 years or older at the time of written informed consent and were required to meet the definition of BCG-unresponsive disease according to the 2018 FDA guidelines. This encompasses patients with persistent CIS or high-grade papillary disease at 6 months despite receiving adequate BCG, which is defined as a minimum of 5 of the 6 induction doses and 2 of the 3 maintenance treatments (previously termed “BCG refractory”). It also includes patients who have recurrence of high-grade papillary NMIBC within 6 months or CIS within 12 months of achieving a disease-free state following BCG (previously termed “BCG relapsed”), as well as patients with persistent or progression to T1 disease after induction BCG. Patients had an Eastern Cooperative Oncology Group (ECOG) status of 2 or less and a life expectancy of at least two years. All visible papillary tumors were required to be resected, and patients with T1 disease on transurethral resection of bladder tumor (TURBT) were required to undergo an additional re-TURBT within 14 to 60 days prior to beginning study treatment. Obvious areas of CIS were also fulgurated prior to beginning study treatment.

### Efficacy Outcomes

Overall, 151 patients were included in the efficacy analysis set (103 in the CIS cohort and 48 in the papillary [Ta/T1] cohort), which included all patients meeting the stringent definition of BCG-unresponsive NMIBC. Six patients (4 in the CIS cohort, 2 in the papillary cohort) were excluded from the efficacy analysis set as they did not meet major study entry criteria, in accordance with ICH E9 guidance. At the time of data analysis cut-off for this report on Jul. 8, 2019, all patients had completed either the Month 12 efficacy assessment visit or had withdrawn from treatment and completed a safety assessment. Further data analysis was performed at the Month 18 and 24 efficacy assessment visits.

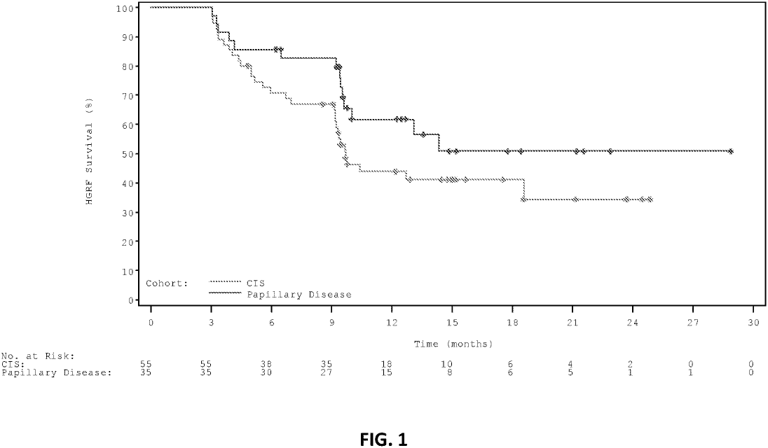

With regard to the study’s primary endpoint, 55 (53.4%) patients (95% CI: [43.3, 63.3]) in the CIS cohort achieved the primary endpoint of a CR, with all CRs noted by Month 3 (Table 2). Median duration of the CR among patients with CIS was 9.69 months (95% CI, 9.17 months to not estimable) (Table 2), with 25 patients (24.3%, 95% CI [16.4, 33.7]) maintaining high-grade RFS at 12 months (FIG. 1). Specifically among CIS patients with an initial CR, 45.5% remained free from high-grade recurrence at 12 months. Among patients with CIS, 73 (70.9%) developed recurrent high-grade NMIBC and five (4.9%) progressed to detrusor muscle invasion (≥pT2). Of the patients that progressed to MIBC, 3 had a prior history of T1 NMIBC at trial entry, 1 recurred with CIS at month 9 and had occult pT2N0 identified at radical cystectomy, and the last recurred with CIS at month 3, was enrolled on the KEYNOTE-057 clinical trial and received systemic pembrolizumab before progressing to pT2N1 at cystectomy.

TABLE 2

Summary of Incidence of High-Grade Recurrence-Free Survival after treatment with ADSTILADRIN®

| | CIS N = 103 | | Papillary disease (N=48) | | Total (N=151) | |
|---|---|---|---|---|---|---|
| Patients who achieved CR | n (%) | 95% CI^{b} | n (%) | 95% CI | n (%) | 95% CI |
| By Month 3 | 55 (53.4) | 43.3, 63.3 | 35 (72.9) | 58.2, 84.7 | 90 (59.6) | 51.3, 67.5 |
| Duration of complete response† or high-grade recurrence-free survival‡ (months) | Median | 95% CI | Median | 95% CI | Median (%) | 95% CI |
| | 9.72 | 9.17, 24.28 | 12.35 | 6.67, 20.27 | 7.31 | 5.68, 11.93 |
| Patients who achieved HGRF at | n (%) | 95% CI | n (%) | 95% CI | n (%) | 95% CI |
| Month 6 | 42 (40.8) | 31.2, 50.9 | 30 (62.5) | 47.4, 76.0 | 72 (47.7) | 39.5, 56.0 |
| Month 9 | 36 (35.0) | 25.8, 45.0 | 28 (58.3) | 43.2, 72.4 | 64 (42.4) | 34.4, 50.7 |
| Month 12 | 25 (24.3) | 16.4, 33.7 | 21 (43.8) | 29.5, 58.8 | 46 (30.5) | 23.2, 38.5 |
| Month 18 | 22 (21.4) | 13.9, 30.5 | 16 (33.3) | 20.4, 48.4 | 38 (25.2) | 18.5, 32.9 |
| Month 24 | 20 (19.4) | 12.3, 28.4 | 13 (27.1) | 15.3, 41.8 | 33 (21.9) | 15.5, 29.3 |

CIS = carcinoma in situ; CI = confidence interval; CR = complete response; NE = not estimable.
[1] The CIS cohort included patients with CIS with and without and concomitant papillary disease (Ta/T1). Patients who achieved CR comprised all patients who had both CR reported by the study investigator and CR according to previously defined criteria. A 95% exact binomial CI for rate of CR was calculated using the Clopper-Pearson method. Median is Kaplan-Meier estimate. The two-sided CI for median was computed using the method of Brookmeyer and Crowley. Probability of duration was the Kaplan-Meier estimate of survivor function at each specific time point. The 95% CI for probability of duration was calculated using Greenwood’s formula with a logarithmic-logarithmic transformation. A patient achieved HGRF survival at a time point if the patient was alive and without documented recurrence of high-grade disease or muscle-invasive disease progression as assessed by the Investigator at that time point.
[2] Note: a patient was judged to have not achieved HGRF survival at the initial Month 3 Efficacy Assessment Visit if urine cytology was reported as suspicious, malignant cells, or not done, and no bladder biopsy was performed at the Month 3 Efficacy Assessment Visit, and there was high-grade recurrence, confirmed by bladder biopsy, observed at the next scheduled visit OR biopsy was not performed at the next visit but urine cytology result at the next scheduled visit was reported as “suspicious” or “malignant.”
†Patients in the carcinoma in situ cohort. ‡Patients in the high-grade Ta or T1 cohort.

Of the patients with high grade papillary (Ta/T1) NMIBC, 35 (72.9%; 95% CI: [58.2, 84.7]) were high-grade recurrence-free at month 3, and 21 patients (43.8%, 95% CI [29.5, 58.8]) remained free of high-grade recurrence at month 12 (FIG. 1). Thus, with regard to durability of response, 60.0% of patients with high grade papillary-only NMIBC who were high-grade recurrence-free at 3 months maintained that status at 12 months. Median duration of HG RFS was 12.35 months (95% CI [6.67, 20.27]). Among this cohort, 23 (48.0%) had a biopsy proven high-grade NMIBC recurrence during follow-up, one (2.1%) died of a non-bladder cancer related cause (with no evidence of recurrence), and three (6.3%) experienced progression to MIBC. Of these 3 patients, two were identified with occult MIBC at radical cystectomy; among the latter, one had recurrence at 9 months with CIS and was found to have pT2bN0 MIBC at time of cystectomy, while the other was found to have pT2aN0 MIBC after recurring with cT1 NMIBC at M12.

Six patients with a normal cystoscopy were found to have CIS at the time of a 12-month biopsy. All patients received a biopsy at 12 months of treatment, although patients showing no recurrence of high-grade disease by cystoscopy or cytology would typically not have a biopsy. These patients were considered to have experienced high-grade tumor recurrence; however, five of these six would in fact have been considered free from high-grade recurrence based on cytology and cystoscopy alone (had the biopsies not been performed, as is typical in the art when cytology and cystoscopy are negative for high-grade tumor recurrence), while one participant did have a suspicious urine cytology. Thus, in the absence of the biopsy, 28 (27.2%) and 23 (47.9%) of CIS and papillary patients, respectively, experienced high-grade recurrence free survival at 12 months.

For the total study cohort, recurrences of any stage were observed in 104 (68.9%) patients, with high-grade NMIBC observed in 96 (61.1%) individuals, progression to muscle-invasive bladder cancer observed in 8 (5.3%) patients, and one on-study non-bladder cancer-related death. High-grade recurrence free survival at one year was 30.5% (95% CI [23.2, 38.5]).

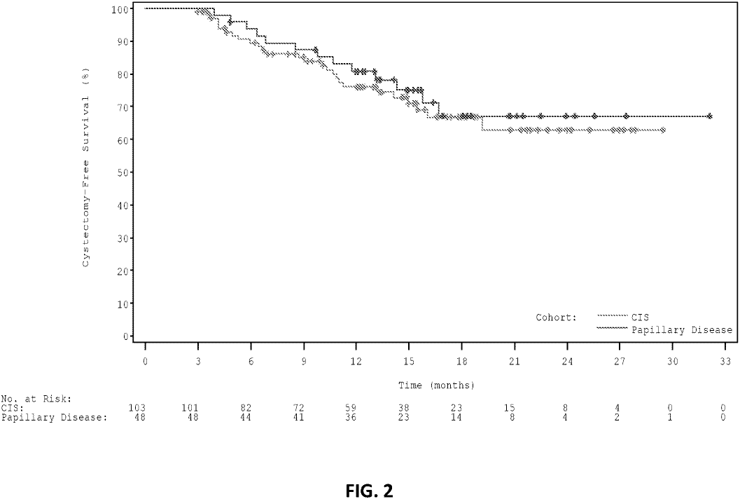

A total of 43 (28.5%) patients underwent cystectomy by the data cut-off, including 32 (31.1%) in the CIS cohort and 11 (22.9%) in the papillary disease cohort. As such, 24-month cystectomy-free survival among all treated patients was 64.5% (95% CI [53.6, 73.4]), and was similar between the cohorts (FIG. 2). Pathologic data was available for all but two patients (one each in the CIS and papillary disease cohorts). Among patients with CIS who underwent cystectomy, one patient was downstaged to pT0 and three were upstaged to pT2 or greater, including one patient with pT2N1 disease who recurred with CIS after ADSTILADRIN®, and progressed after treatment with pembrolizumab on Keynote-057. Two of these 3 patients also had a remote history of cT1 NMIBC before entering the trial with CIS. Among patients in the papillary cohort who underwent cystectomy, four (36.4%) were found to have divergent pathology with CIS present in the cystectomy specimen, and 2 were identified with occult MIBC. Thus, only 5 (11.5%) patients were upstaged to muscle-invasive or extravesical disease at cystectomy.

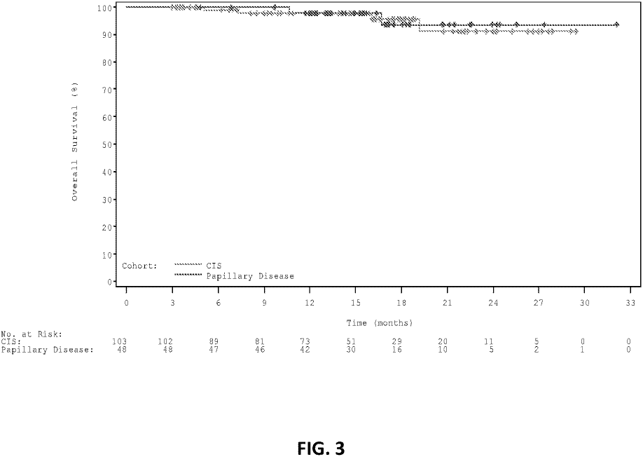

The 24-month OS among all patients who received at least one dose of ADSTILADRIN® was 91.9% (95% CI [80.9, 96.7]), 91.2% (95% CI [74.7, 97.1]) among patients with CIS, and 93.5% (95% CI [75.0, 98.5]) among patients with papillary disease (FIG. 3). Five patient deaths were reported in patients who were in long-term follow-up and off treatment; 3 secondary to a cardiac event and one from pneumonia. The cause of death was unknown for one patient. All 5 deaths occurred at least 4 months after the last administration of the study drug.

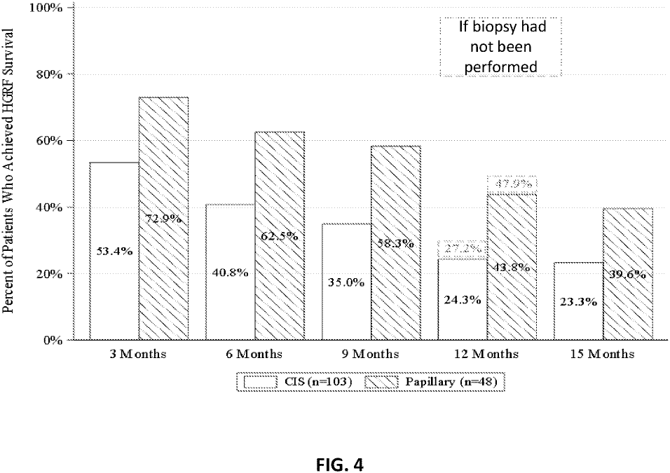

Of those patients with CIS that demonstrated a complete response, 42 (76.4%), 36 (65.5%), 25 (45.5%), 24 (43.6%), 22 (21.4%), and 20 (19.4%) remained high grade recurrence free at 6, 9, 12, 15, 18, and 24 months, respectively (Table 3 and FIG. 4). Of those papillary patients who achieved high-grade recurrence free survival at 3 months, 30 (85.7%), 28 (80.0%), 21 (60.0%), 19 (54.3%), 16 (33.3%), and 13 (27.1%) remained high-grade recurrence free at 6, 9, 12, 15, 18, and 24 months, respectively. The median durability of complete response was estimated at 9.72 months (9.17-24.28) based on 12 month data.

TABLE 3

Summary of Incidence of High-Grade Recurrence-Free Survival after treatment with ADSTILADRIN®

| | CIS (N=103) | Papillary (N=48) | All patients (N = 151) |
|---|---|---|---|
| Patients achieving high-grade recurrence-free survival* at | | | |
| 3 months (n, %) 95% CI [%] | 55 (53.4) [43.3, 63.3] | 35 (72.9) [58.2, 84.7] | 90 (59.6%) [51.3- 67.5] |
| 6 months (n, %) 95% CI [%] | 42 (40.8) [31.2, 50.9] | 30 (62.5) [47.4, 76.0] | 72 (47.7%) [39.5- 56.0] |
| 9 months (n, %) 95% CI [%] | 36 (35.0) [25.8, 45.0] | 28 (58.3) [43.2, 72.4] | 64 (42.4%) [34.4- 50.7] |
| 12 months (n, %) 95% CI [%] | 25 (24.3)** [16.4, 33.7] | 21 (43.8)** [29.5, 58.8] | 46 (30.5%) [23.2- 38.5] |
| 15 months (n, %) 95% CI [%] | 24 (23.3) [15.5, 32.7] | 19 (39.6) [25.8, 54.7] | |
| 18 months (n, %) 95% CI [%] | 22 (21.4) [13.9- 30.5] | 16 (33.3) [20.4- 48.4] | 38 (25.2) [18.5- 32.9] |
| 24 months (n, %) 95% CI [%] | 20 (19.4) [12.3- 28.4] | 13 (27.1) [15.3- 41.8] | 33 (21.9) [15.5- 29.3] |

*Percentage is calculated using the number of patients in the column heading as the denominator. A total of 151 patients were eligible for the efficacy analysis, as six patients were not BCG unresponsive.
** In the absence of biopsies at Month 12, the number of patients with high-grade recurrence free survival would have been: 28 (27.2%) in the CIS cohort and 23 (47.9%) in the papillary cohort.

### Safety Outcomes

Patient demographic information and study drug-related adverse events (occurring in ≥ 5% patients during the study) are shown in Tables 4 and 5. Overall, 146 patients (93.0%) experienced adverse events (AEs) during the study; of these, 110 patients (70.1%) experienced events that were study drug-related (Tables 4 and 5). The most frequently reported drug-related AEs were discharge around the catheter during instillation (n=39, 24.8%), fatigue (n=31, 19.7%), bladder spasms (n=25, 15.9%), and micturition urgency (n=24, 15.3%) (Table 3). Notably, for the majority of patients, the AEs were transient and were classified as either grade 1 or 2. Grade 3 or 4 adverse events occurred in 29 patients (18.5%), of which only 6 patients (3.8%) experienced events that were study drug-related; these included 2 cases of micturition urgency, and 1 case each of bladder spasms, urinary incontinence, syncope, and hypertension. A grade 4 event of sepsis was considered study drug-unrelated but was procedure-related. There were no grade 5 AEs. Three patients discontinued the study drug due to AEs: one patient each for bladder spasms, discharge around the catheter during instillation, and the identification of a benign neoplasm of the bladder.

TABLE 4

Patient demographic information

| Characteristics | CIS N = 107 | Papillary disease N= 50 | Total N = 157 |
|---|---|---|---|
| Age at baseline (years); median (IQR) | 72.0 (66.0, 77.0) | 71.0 (64.0, 78.0) | 71.0(66.0, 77.0) |
| Sex (n, %) | | | |
| Male | 95 (88.8) | 34 (68.0) | 129 (82.2) |
| Female | 12 (11.2) | 16 (32.0) | 28 (17.8) |
| Race | | | |
| White | 99 (92.5) | 47 (94.0) | 146 (93.0) |
| Black or Africa American | 6 (5.6) | 2 (4.0) | 8 (5.1) |
| Asian | 2 (1.9) | 1 (2.0) | 3 (1.9) |
| Time from Initial Diagnosis of Bladder Cancer (Months); median Number of Prior Courses of BCG Administered | 20.04 | 14.72 | 18.04 |
| 1 | 1 (0.9) | 5 (10.0) | 6 (3.8) |
| 2 | 45 (42.1) | 28 (56.0) | 73 (46.5) |
| 3 | 28 (26.2) | 12 (24.0) | 40 (25.5) |
| 4 | 12 (11.2) | 2 (4.0) | 14 (8.9) |
| ≥5 | 21 (19.6) | 3 (6.0) | 24 (15.3) |
| Stage at entry | | | |
| CIS only | 81 (75.7) | 0 (0.0) | 81 (51.6) |
| Ta | 0 (0.0) | 35 (70.0) | 35 (22.3) |
| Ta + CIS | 21 (19.6) | 0 (0.0) | 21 (13.4) |
| T1 | 0 (0.0) | 15 (30.0) | 15 (9.6) |
| T1 + CIS | 5 (4.7) | 0 (0.0) | 5 (3.2) |
| ECOG status | | | |
| 0 | 97 (90.7) | 43 (86.0) | 140 (89.2) |
| 1 | 7 (6.5) | 6 (12.0) | 13 (8.3) |
| 2 | 3 (2.8) | 1 (2.0) | 4 (2.5) |

BCG = Bacillus Calmette-Guerin; CIS = carcinoma in situ; ECOG = Eastern Cooperative Oncology Group; SD = standard deviation. The CIS cohort included patients with CIS with and without and concomitant papillary disease (Ta/T1). Baseline was defined as the last measurement obtained prior to the first administration of the study drug.

TABLE 5

Drug-related adverse events in patients

| | Total N = 157 n (%) | Total N = 157 n (%) |
|---|---|---|
| | ALL GRADES | CTCAE Grades 3-4*† |
| Patients with any study drug-related adverse event | 110 (70.1) | |
| Patients with any study drug-related grade 3-5 adverse event | | 6 (3.8) |
| General disorders and administration site conditions | 76 (48.4) | 0 (0.0) |
| Discharge around the catheter during instillation | 39 (24.8) | 0 (0.0) |
| Fatigue | 31 (19.7) | 0 (0.0) |
| Chills | 18 (11.5) | 0 (0.0) |
| Pyrexia | 16 (10.2) | 0 (0.0) |
| Pain | 10 (6.4) | 0 (0.0) |
| Renal and urinary disorders | 68 (43.3) | 4 (2.5) |
| Bladder spasm | 25 (15.9) | 1 (0.6) |
| Micturition urgency | 24 (15.3) | 2 (1.3) |
| Dysuria | 17 (10.8) | 0 (0.0) |
| Pollakiuria | 10 (6.4) | 0 (0.0) |
| Bladder pain | 8 (5.1) | 0 (0.0) |
| Gastrointestinal disorders | 21 (13.4) | 0 (0.0) |
| Diarrhea | 10 (6.4) | 0 (0.0) |
| Nervous system disorders | 21 (13.4) | 1 (0.6) |
| Headache | 14 (8.9) | 0 (0.0) |
| | 10 (6.4) | 0 (0.0) |

*There were no grade 5 study drug-related adverse events.
†Other CTCAE grade 3-4 study drug-related adverse events were: Syncope (1 patient [0.6%]), hypertension (1 [0.6]), and urinary incontinence (1 [0.6]).
CIS = carcinoma in situ.
The CIS cohort included patients with CIS with and without and concomitant papillary disease (Ta/T1). Adverse events include all events that occurred or worsened after the first dose of rAd-IFNα/Syn3.

### Conclusion

In the present study, intravesical ADSTILADRIN® achieved a complete response in 53.4% of patients with BCG-unresponsive CIS±Ta/T1, the primary analysis population. This greatly exceeds the CR rate achieved by valrubicin in two separate studies and represents a clinically meaningful improvement. Furthermore, CRs appeared to be encouragingly durable, with nearly half of patients who achieved a CR remaining free of high-grade recurrence at 12 months. Among patients with papillary BCG-unresponsive NMIBC, the CR rate at 3 months was 72.9%. Importantly, progression to muscle invasion was a rare event, occurring in only 5 patients (4.9%) in the CIS cohort and 3 patients (6.3%) of the papillary cohort. These rates of progression are lower than have been previously reported for patients with BCG unresponsive NMIBC treated with bladder sparing therapy, suggesting that instillation of ADSTILADRIN® did not put patients at higher risk for progression and subsequent death from bladder cancer, and might alter the natural history of the disease. Moreover, the cystectomy-free survival at 24 months for the total study cohort was 64.5%, indicating that the majority of patients with this difficult to treat entity had still elected to avoid the morbidity of surgery.

Among patients who underwent cystectomy, 3/32 (9.3%) in the CIS cohort were found to have pT2 or higher stage disease, and this included one patient who had been treated with and had progressed on pembrolizumab before proceeding to cystectomy. Two of the 3 patients had a remote history of cT1 disease before entering the trial suggesting that these patients may have been understaged at the time of screening. Additionally, several patients in the papillary cohort undergoing cystectomy were found to have concomitant CIS on final pathology (4/11; 36.3%). The presence of this divergent pathology within the papillary cohort highlights the prevalence of CIS at the time of cystectomy. It is likely that many patients with papillary disease also had occult CIS at initial diagnosis, left undetected until the time of cystectomy. Historically, patients undergoing a resection of papillary tumor have also undergone random bladder biopsies to identify occult CIS, but this is no longer routinely performed since the results of such biopsies do not impact treatment.

Additionally, bladder biopsies at the 12-month time point served to ensure the veracity of the CRs observed prior to commencing longer term follow-up and durability assessments. The safety profile of ADSTILADRIN® here was acceptable, with only 3 patients stopping treatment due to an adverse event, no treatment-related deaths, and no pattern of immune-related adverse events noted. Moreover, the dosing schedule of ADSTILADRIN® -one intravesical treatment every three months -was convenient for both patients and physicians.

In summary, intravesical ADSTILADRIN® for patients with BCG-unresponsive NMIBC demonstrated first-of-its-kind efficacy for gene therapy as well as a manageable safety profile and delivery schedule, resulting in a favorable benefit-risk profile. These data support ADSTILADRIN® as a significant therapeutic advancement for a historically difficult to treat disease state.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a high-grade non-muscle-invasive bladder cancer (NMIBC) in a subject, comprising administering a composition to the subject intravesically every three months for at least one year, wherein the composition comprises: (i) a non-replicating recombinant adenoviral vector (e.g., a type 5 non-replicating recombinant adenoviral vector) encoding interferon α-2b (IFN α-2b), and (ii) [N-(3-cholamidopropyl)-N-(3-lactobionamidopropyl)]-cholamide (SYN3), thereby treating NMIBC in the subject.

2. The method of claim 1, further comprising performing an assay on the subject or on a sample from the subject, e.g., at about one year or longer after administration of the composition.

3. The method of claim 2, wherein the assay indicates efficacy of the composition.

4. A method of evaluating or monitoring efficacy of a composition in a subject having or diagnosed with high-grade non-muscle-invasive bladder cancer (NMIBC), comprising performing an assay on the subject or on a sample from the subject,
wherein the composition comprises: (i) a non-replicating recombinant adenoviral vector encoding interferon α-2b (e.g., a type 5 non-replicating recombinant adenoviral vector), and (ii) [N-(3-cholamidopropyl)-N-(3-lactobionamidopropyl)]-cholamide (SYN3), and
wherein the subject has received intravesical administration of the composition every three months for at least one year.

5. The method of claim 4, further comprising administering the composition to the subject.

6. The method of any one of claims 2-5, further comprising acquiring the sample from the subj ect.

7. The method of any one of claims 2-6, wherein the sample comprises a blood sample (e.g., a plasma or serum sample), saliva sample, tissue sample, or urine sample.

8. The method of any one of claims 2-7, wherein the assay comprises performing cytology on the sample and/or cystoscopy on the subject.

9. The method of any one of claims 2-8, wherein cytology and/or cystoscopy are not performed.

10. The method of any one of claims 1-9, further comprising administering to the subject at least one additional therapeutic agent.

11. The method of claim 8, wherein the at least one additional therapeutic agent comprises a checkpoint inhibitor.

12. The method of claim 11, wherein the checkpoint inhibitor targets a checkpoint molecule chosen from PD-1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (e.g., TNFRSF14 or CD270), KIR, A2aR, MHC class I, MHC class II, GAL9, adenosine, or TGF (e.g., TGF beta).

13. The method of claim 10, wherein the checkpoint inhibitor comprises or is an anti-PD-1 antibody or a fragment thereof, e.g., Pembrolizumab.

14. The method of claim 11, wherein Pembrolizumab is administered intravenously about every three weeks or longer than every three weeks (e.g., every four weeks, every five weeks, every six weeks, or more).

15. The method of any one of claims 1-14, wherein the subject has a complete response (CR), a partial response, or a non-response to treatment with the composition.

16. The method of any one of claims 1-15, wherein the subject does not exhibit high-grade recurrence after receiving treatment with the composition, e.g., for at least one year after administration of the composition.

17. The method of any one of claims 1-16, wherein the subject has an Eastern Cooperative Oncology Group status of 2 or less than 2.

18. The method of any one of claims 1-17, wherein the patient has high-risk NMIBC.

19. The method of any one of claims 1-18, wherein the patient has not been responsive to at least one prior treatment comprising Bacillus Calmette-Guerin (BCG).

20. The method of claim 19, wherein the subject has received more than one treatment with BCG.

21. The method of claim 19 or 20, wherein the subject exhibited recurrence of NMIBC after at least one prior treatment comprising BCG.

22. The method of any one of claims 1-21, wherein the subject has BCG refractory NMIBC.

23. The method of any one of claims 1-22, wherein the subject has relapsed NMIBC.

24. The method of any one of claims 1-23, wherein the subject has or has been diagnosed with one or both of carcinoma in situ (CIS) or high-grade papillary disease.

25. The method of any one of claims 1-24, wherein the subject has or has been diagnosed with T1 bladder cancer, e.g., after at least one prior treatment comprising BCG.

26. The method of any one of claims 1-25, wherein the subject has previously received at least one treatment comprising pembrolizumab, valrubicin, gemcitabine, docetaxel, gemcitabine and mitomycin in combination, gemcitabine and docetaxel in combination, and/or nab-paclitaxel.

27. The method of any one of claims 1-26, wherein the vector is targeted to bladder epithelium tissue.

28. The method of any one of claims 1-27, wherein the subject receives or has received treatment with an anti-cholinergic agent prior to treatment with the composition.

29. The method of any one of claims 1-28, wherein the non-replicating recombinant adenoviral vector comprises or is a type 5 non-replicating adenoviral vector.

30. The method of any one of claims 1-29, wherein the non-replicating recombinant adenoviral vector is administered at a dose of about 2.25 × 10¹³ viral particles (vp).

31. The method of claim 30, wherein the total volume of the dose is about 75 ml.

32. The method of any one of claims 1-31, wherein the non-replicating recombinant adenoviral vector is administered at a dosage of about 1 × 10¹¹ viral particles(vp)/ml to about 3 × 10¹¹ vp/ml.

33. The method of any one of claims 1-32, wherein the composition is administered or has been administered to a population of subjects.

34. The method of claim 33, further comprising evaluating or monitoring a complete response (CR) in the population of subjects.

35. The method of claim 33 or 34, further comprising evaluating or monitoring, in the population of subjects, one, two, three, or four of: durability of CR (e.g., median or mean durability of CR), high-grade recurrence-free survival, radical cystectomy free survival, or overall survival.

36. The method of any one of claims 33-35, wherein the population of subjects comprises subjects having or diagnosed with one or both of CIS or high-grade papillary disease.

37. The method of any one of claims 33-36, wherein about 24.3% of the subjects (e.g., subjects with CIS) exhibit high-grade recurrence free survival (RFS) after at least about one year of treatment with the composition.

38. The method of any one of claims 33-37, wherein durability of CR (e.g., median or mean durability of CR) for the subjects (e.g., subjects with CIS) is about 9.69 months.

39. The method of any one of claims 33-38, wherein about 45.5% of the subjects (e.g., subjects with CIS) do not exhibit high grade recurrence after at least about one year of treatment with the composition (e.g., after at least about one year following treatment with at least one dose of the composition).

40. The method of any one of claims 33-39, wherein about 43.8% of the subjects (e.g., subjects with papillary disease) exhibit high-grade RFS after at least about one year of treatment with the composition (e.g., after at least about one year following treatment with at least one dose of the composition).

41. The method of any one of claims 33-40, wherein durability of CR (e.g., median or mean durability of CR) in the subjects (e.g., subjects with papillary disease) is about 12.35 months.

42. The method of any one of claims 33-41, wherein about 60% of the subjects (e.g., subjects with papillary disease) do not exhibit high grade recurrence after at least about one year of treatment with the composition (e.g., after at least about one year following treatment with at least one dose of the composition).

43. The method of any one of claims 33-42, wherein durability of CR (e.g., median or mean durability of CR) in the subjects (e.g., subjects with CIS and subjects with papillary disease) is about 7.31 months.

44. The method of any one of claims 33-43, wherein about 30.5% of the subjects (e.g., subjects with CIS and subjects with papillary disease) exhibit high-grade RFS after at least about one year of treatment with the composition (e.g., after at least about one year following treatment with at least one dose of the composition).

45. The method of any one of claims 33-44, wherein cystectomy-free survival of the subjects (e.g., subjects with CIS and subjects with papillary disease) is about 64.5% after at least about two years of treatment with the composition (e.g., after at least about two years following treatment with at least one dose of the composition).

46. The method of any one of claims 33-45, wherein overall survival of the subjects (e.g., subjects with CIS) is about 91.9% after at least about two years of treatment with the composition (e.g., after at least about two years following treatment with at least one dose of the composition).

47. The method of any one of claims 33-46, wherein overall survival of the subjects (e.g., subjects with papillary disease) is about 93.5% after at least about two years of treatment with the composition (e.g., after at least about two years following treatment with at least one dose of the composition).

48. The method of any one of claims 33-47, wherein less than about 4.9% of the subjects (e.g., subjects with CIS) exhibit progression to muscle invasion.

49. The method of any one of claims 33-48, wherein less than about 6.3% of the subjects (e.g., subjects with papillary disease) exhibit progression to muscle invasion.

50. The method of any one of claims 33-49, wherein about 23.3% of the subjects (e.g., subjects with CIS) exhibit high-grade recurrence free survival (RFS) after at least about 15 months of treatment with the composition (e.g., after at least about 15 months following treatment with at least one dose of the composition).

51. The method of any one of claims 33-50, wherein about 39.6% of the subjects (e.g., subjects with papillary disease) exhibit high-grade recurrence free survival (RFS) after at least about 15 months of treatment with the composition (e.g., after at least about 15 months following treatment with at least one dose of the composition).
